(19) United States
(12) Patent Application Publication
(10) Pub. No.: US 2008/0188803 A1
(43) Pub. Date: Aug. 7, 2008

(54) Triple-profile balloon catheter

(76) Inventor: G. David Jang, Redlands, CA (US)

Correspondence Address: HELLER EHRMAN LLP, 275 MIDDLEFIELD ROAD, MENLO PARK, CA, 94025-3506, US

(21) Appl. No.: 11/336,416

(22) Filed: Jan. 19, 2006

Related U.S. Application Data
(60) Provisional application No. 60/650,745, filed on Feb. 3, 2005.

(51) Int. Cl. A61M 25/098; A61M 25/10

(52) U.S. Cl. 604/103.1; 604/96.01

(57) ABSTRACT

A triple profile balloon catheter has a catheter shaft with an inflation/deflation lumen. A balloon is located at a distal section of the catheter shaft. The balloon has a first segment in a proximal portion of the balloon, and a second segment in a distal portion of the balloon. An intermediate segment with a length greater than 3 mm couples the first and second segments. The first segment has a first average diameter, the second segment has a second average diameter that is less than the first average diameter. The balloon has a single chamber coupled to the inflation/deflation lumen.

### CROSS-REFERENCE TO RELATED APPLICATIONS

This application claims the benefit of U.S. Ser. No. 60/650,745 filed Feb. 3, 2005, which application is fully incorporated herein by reference.

### BACKGROUND OF THE INVENTION

1. Field of Invention

This invention relates to generally to balloon catheters, and more particularly to a triple profile balloon catheter for angioplasty applications in the long vessel segment with obstructive disease and for bifurcation or side-branch origin applications.

2. Description of the Related Art

By 2004, interventional angioplasty and stent implant procedures have become the dominant non-surgical revascularization method of the atherosclerotic stenoses of the vascular lumen, particularly in the coronary vascular system. With balloon angioplasty alone, without use of stent, the restenosis rate after angioplasty has been as high as 25-35% in the first time clinical cases. With use of bare metal stents in conjunction with balloon angioplasty, the restenosis was reduced significantly. Even so, the restenosis rate after stent implant is reported as a 10-20% range depending on the condition of a vessel stented or what specific stent brand was used, requiring a need for further restenosis reducing measures after intravascular stenting.

To further reduce the restenosis rate after stent implant, numerous means designed to reduce restenosis rate has been tried, including laser, atherectomy, high frequency ultrasound, radiation device, local drug delivery, etc. Although the brachytherapy (radiation treatment) has proved to be somewhat effective early in reducing restenosis after stent implant, the long term follow up results were not very encouraging and using the brachytherapy is cumbersome, inconvenient and costly. Mainly because it is a radioactive device with a declining isotope half-life, and radiation therapy specialist from another department has to be involved with each procedure. The laser and atherectomy devices proved to be marginally useful in this purpose with added costs.

By 2003, drug-coated or drug-eluting stents have been introduced into the U.S. market after an FDA approval. The first U.S. approved drug-eluting stent has Sirolimus, an immune-suppressive drug, as main agent as anti-restenosis. This stent has further reduced a medium term restenosis down to 5% range. A cancer treatment drug, Paclitaxol, coated stent is also introduced in the U.S. in 2004 with a remarkable success. Both of these drug-eluting stents has changed dramatically the restenosis rate after coronary stent implants.

With these promising restenosis rate improvements made with the drug-eluting stents, potential prospect for angioplasty and stent implant in the vessels associated with the bifurcation or side branch anatomy has also improved. However, successful strategy for angioplasty and stenting of the vessels associated with bifurcation or side-branch requires two very fundamental elements.

There is a need for a specially designed stent that will readily adapt to a set of complex anatomic characteristics of a coronary artery lesion at a bifurcation or side-branch origin, which is far more complex and difficult for a stent to optimally adapt to. A stent that is designed for a regular vessel that is basically a single lumen tubular structure, cannot adopt to a multi-lumen and multi-diameter bifurcation lesions. The next requirement is a specially designed angioplasty-stent delivery balloon catheter that is adoptable to the complex anatomic characteristics of a bifurcation or side-branch origin lesions. A specially designed stent cannot be effectively used if there is no specially designed angioplasty-stent delivery balloon catheter that is adapted to the anatomic characteristics of a bifurcation or side-branch origin lesions of coronary artery.

There is a need for a specially designed balloon catheter system for the bifurcation or side-branch origin applications.

### SUMMARY OF THE INVENTION

Accordingly, an object of the present invention is to provide an improved balloon catheter.

Another object of the present invention is to provide a balloon catheter for both angioplasty and stent delivery.

A further object of the present invention is to provide a balloon catheter for bifurcation and side branch anatomies.

Still another object of the present invention is to provide a triple profile balloon catheter for the vessels associated with bifurcation and side branch anatomies.

A further object of the present invention is to provide a triple profile balloon catheter for vessel dilatation in diseased vessels associated with bifurcation and side branch anatomies.

Yet another object of the present invention is to provide a triple profile balloon catheter for use in diseased vessels associated with bifurcation and side branch anatomies that minimizes complications or undesirable side effects.

Yet another object of the present invention is to provide a safe and anatomically designed balloon catheter that matches the anatomy in a long diseased segment of a vessel that has a larger proximal diameter and a smaller distal diameter.

These and other objects of the present invention are achieved in a triple profile balloon catheter that has a catheter shaft with an inflation/deflation lumen. A balloon is located at a distal section of the catheter shaft. The balloon has a first segment in a proximal portion of the balloon, and a second segment in a distal portion of the balloon. An intermediate segment with a length greater than 3 mm couples the first and second segments. The first segment has a first average diameter, the second segment has a second average diameter that is less than the first average diameter. The balloon has a single chamber coupled to the inflation/deflation lumen.

In another embodiment of the present invention, a triple profile balloon catheter has a catheter shaft with an inflation/deflation lumen and a guidewire lumen. A balloon is located at a distal section of the catheter shaft. The balloon has a first segment in a proximal portion of the balloon, and a second segment in a distal portion of the balloon. The first and second segments are coupled by an intermediate segment with a length greater than 3 mm. The first segment has a first average diameter, and the second segment has a second average diameter that is less than the first average diameter. A first radiopaque marker is positioned at or near a transition junction between the first segment and the intermediate segment of the balloon.

In another embodiment of the present invention, a triple profile balloon catheter has a catheter shaft with an inflation/deflation lumen. A balloon is located at a distal section of the catheter shaft. The balloon has a first segment in a proximal portion of the balloon, and a second segment in a distal portion of the balloon. The first and second segments are coupled by an intermediate segment with a length greater than 3 mm. The first segment has a first average diameter, and the second segment has a second average diameter that is less than the first average diameter. A second radiopaque marker is positioned at a transition junction between the second segment and the intermediate segment of the balloon.

In another embodiment of the present invention, a triple profile balloon catheter has a catheter shaft with an inflation/deflation lumen. A balloon is located at a distal section of the catheter shaft. The balloon has a first segment in a proximal portion of the balloon, and a second segment in a distal portion of the balloon. The first and second segments are coupled by an intermediate segment. The first segment has a first average diameter, and the second segment has a second average diameter that is less than the first average diameter. A first radiopaque marker is positioned at or near a transition junction between the first segment and the intermediate segment. A second radiopaque marker is positioned at or near a transition junction between the second segment and the intermediate segment of the balloon.

In another embodiment of the present invention, a triple profile balloon catheter has a catheter shaft with an inflation/deflation lumen and a guidewire lumen. A balloon is located at a distal section of the catheter shaft. The balloon has a first segment in a proximal portion of the balloon, and a second segment in a distal portion of the balloon. The first and second segments are coupled by an intermediate segment. The first segment has a first average diameter, and the second segment has a second average diameter that is less than the first average diameter. The balloon has a single chamber coupled to the inflation/deflation lumen. At least a first radiopaque marker is positioned at or near a transition junction between the first segment and the intermediate segment or positioned at or near a transition junction between the second segment and the intermediate segment of the balloon.

In another embodiment of the present invention, a triple profile balloon catheter has a catheter shaft with an inflation/deflation lumen and a guidewire lumen. A balloon is located at a distal section of the catheter shaft. The balloon has a first segment in a proximal portion of the balloon, and a second segment in a distal portion. of the balloon. The first and second segments are coupled by an intermediate segment. The first, second and intermediate segments each have a different profile. The balloon has a single chamber coupled to the inflation/deflation lumen.

In another embodiment of the present invention, a triple-profile balloon catheter has a single inner chamber and a catheter shaft with an inflation/deflation lumen and a guidewire lumen. A balloon is located at a distal section of the catheter shaft. The balloon has a first segment in a distal portion of the balloon, and a second segment in a proximal portion of the balloon. The first and second segments are coupled by an intermediate segment with a length greater than 3 mm. The first segment has a first average diameter, and the second segment has a second average diameter that is less than the first average diameter. A first radiopaque marker is positioned at or near a transition junction between the first segment and the intermediate segment of the balloon.

In another embodiment of the present invention, a triple profile balloon catheter is provided that includes a catheter shaft with an inflation/deflation lumen and a guidewire lumen. A balloon is located at a distal section of the catheter shaft. The balloon has a first segment in a distal portion of the balloon, and a second segment in a proximal portion of the balloon. The first and second segments are coupled by an intermediate segment. The first, second and intermediate segments each have a different profile, the balloon having a single chamber coupled to the inflation/deflation lumen.

### BRIEF DESCRIPTION OF THE DRAWINGS

FIG. 1 is a schematic diagram of one embodiment of a triple profile balloon catheter of the present invention in an expanded side view.

FIG. 2 is similar to the FIG. 1 embodiment and illustrates three segments that are demarcated with vertical dotted lines, and two radiopaque markers.

FIG. 3(a) is a schematic illustration showing the longitudinal cross-section of the FIG. 1 triple profile balloon catheter in an inflated profile and with a stent mounted on exterior to the balloon.

FIG. 3(b) is a longitudinal cross-section view of the expanded stent from FIG. 3(a). The vertical dotted lines indicate the three profile zones of the inflated balloon and the expanded stent, which is expanded and molded by the inflated balloon underneath.

FIG. 4 is a side elevation view of the deflated and folded balloon of the FIG. 1 embodiment.

FIG. 5 is a schematic illustration of a deflated and folded triple-profile balloon, of the present invention, with a stent crimp-mounted there over.

FIG. 6 is a schematic illustration of a rapid-exchange catheter system for a triple-profile angioplasty balloon of the present invention.

FIG. 7 is a schematic illustration of an over-the-wire catheter system for a triple-profile angioplasty balloon, of the present invention, with a stent mounted on the balloon for delivery.

### DETAILED DESCRIPTION

In one embodiment of the present invention, a triple profile balloon catheter 10, of the present invention, has a catheter shaft 26 with an inflation/deflation lumen 32. A triple profile balloon 100 is located at a distal section of the catheter shaft 26. The balloon 100 has a first segment 110 in a proximal portion of the balloon 100, and a second segment 112 in a distal portion of the balloon 100. The first and second segments being 110 and 112 are coupled by an intermediate segment 114 with a length greater than 3 mm. The first segment 110 has a first average diameter. The second segment 112 has a second average diameter. In one embodiment, the second average diameter is less than the first average diameter. In one embodiment, the balloon 100 has a single chamber, generally denoted as 40 is connected to the inflation/deflation aperture 24 which is connected to the main inflation/deflation lumen 32. A first radiopaque marker 120 can be provided. The first radiopaque marker 120 can be located at or near, the transition junction between the first segment 100 and the intermediate segment 114 inside of the balloon chamber 40, and a second radiopaque marker 122 can be located at or near the transition junction between the second segment 112 and the intermediate segment 114 inside the balloon chamber 40. The radiopaque markers 124 and 126 can be positioned at a proximal end of the balloon 100 and at a distal end of the balloon 100, respectively.

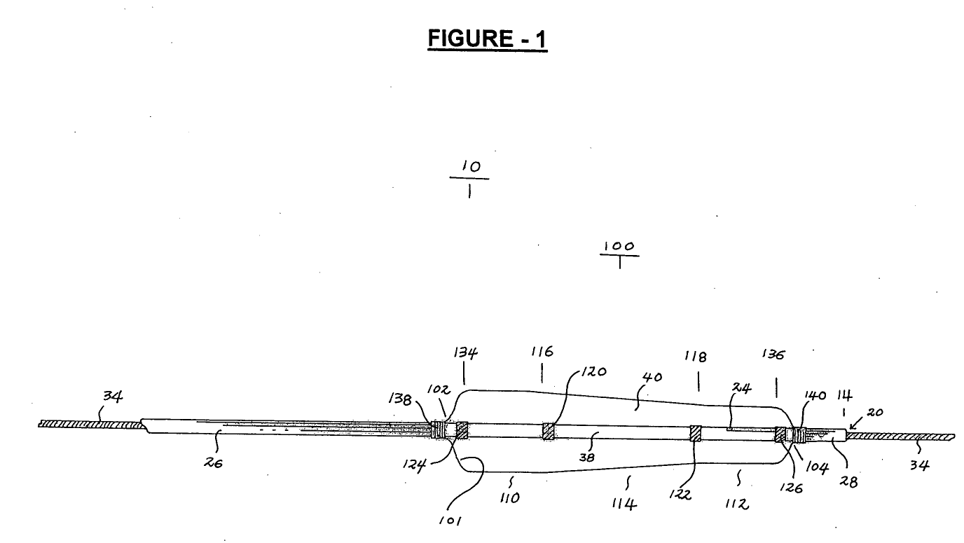

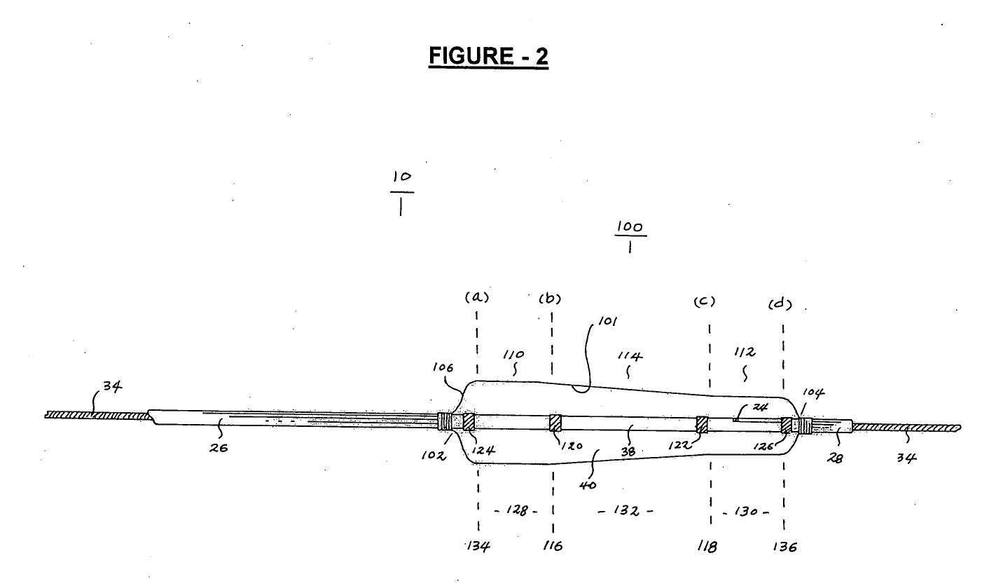

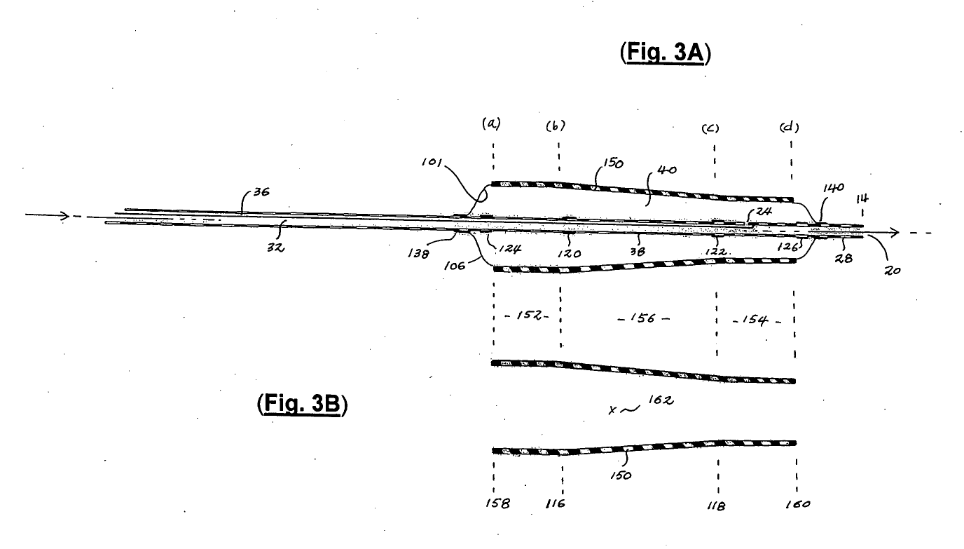

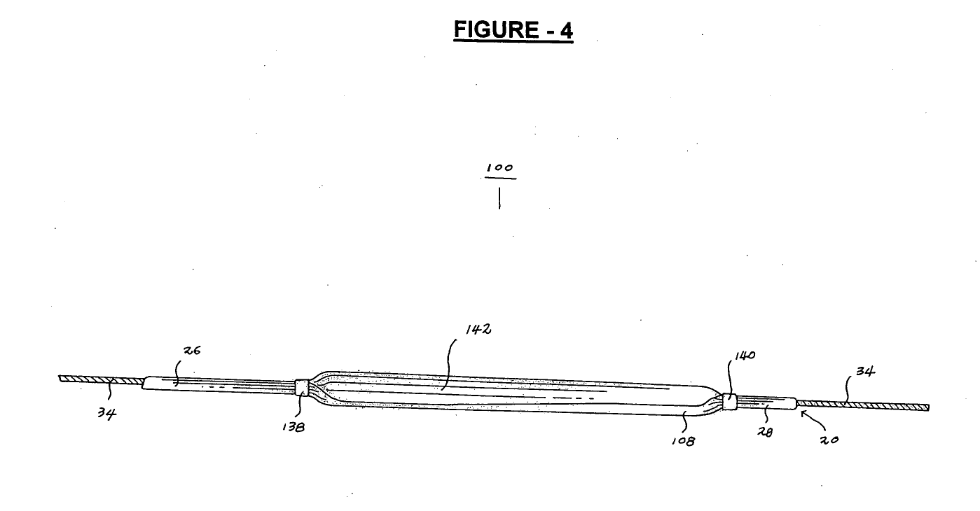

In various embodiments, the balloon 100 has a smooth transition junction between the first segment 110 and the intermediate segment 114, a smooth transition junction between the intermediate segment 114 and the second segment 112. For a smooth transition silhouette between the first and second diameters, the intermediate segment 114 has a straight transition silhouette between the first 110 and second 112 diameters when the balloon is nominally inflated and the intermediate segment 114 has a non-parallel transition silhouette between the first and second diameters when the balloon is nominally inflated. The triple profile balloon catheter 100 is depicted in an inflated side elevation view in FIGS. 1 and 2, and illustrates a longitudinal cross-section in FIG. 3(a). A folded-balloon in side elevation view is in FIG. 4, and a stent-mounted over a folded balloon in FIG.-5.

Further describing the total catheter 10 embodiment, FIG. 1 contains; the catheter shaft 38 inside balloon chamber 40, the inflation/deflation lumen 32 aperture 24, the catheter distal shaft 28 has a distal end 14 with the distal guidewire opening 20 and a guidewire 34 protruding, the proximal 102 and distal 104 ends of the balloon 100 closely associated with the proximal 138 and distal 140 balloon bonding joints, the proximal end 134 and the distal end 136 marks the effective length of the balloon 100, the marker points 116 and 118, respectively, indicate the first segment 110-intermediate segment 114 junction and the second segment 112-intermediate segment 114 junction. In this schematic inflated balloon 100, the balloon skin 101 defines the silhouette of the triple-profile shape of the balloon 100.

Further describing balloon 100, FIG.-2 shows vertical dotted lines are the marking points 116(a), 118(b), 134 (c) and 136 (d). These four lines define the three zones; the proximal first segment 110 represents the proximal parallel zone 128, distal second segment 112 represents distal parallel zone 130 and the intermediate segment 114 represents the intermediate zone 132. In the forgoing descriptions, the proximal zone 128 and distal zone 130 does not have to be a parallel silhouette, and this variation are within the scope of the present invention.

To further describe the design details of the triple-profile balloon 100, FIG.-3A and FIG.-3B shows the longitudinal cross section of the catheter 10 and the balloon 100 in an inflated profile, with an expanded stent 150 mounted over the outer side of the balloon 100. Note that the silhouette of the expanded stent 150 is closely shaped and molded after the silhouette of the expanded profile of the balloon 100. The three zones, proximal 152, distal 154 and intermediate 156, are all shaped and molded after the same zones (152,154 & 156) of the expanded balloon 100. The radiopaque markers 120, 122, 124 and 126 corresponds with the balloon 100 profile demarcation lines 158(a), 160(d), 116(b) and 118(c). In the catheter shaft 26, there is the guidewire lumen 32 with the distal opening 20 at distal end 14 of the distal shaft segment 28, which does not have an inflation/deflation lumen 36. The catheter shaft 26 also contains an inflation/deflation lumen 36, which ends at the balloon shaft 38 and the inflation/deflation aperture 24 inside the balloon chamber 40. When the balloon 100 is deflated and withdrawn from the expanded stent 150, the stent has a triple-profile silhouette and an open lumen 162. The catheter 10 in FIG.-4 has a folded balloon skin 108 forming a low profile balloon 142 for insertion into a vessel lumen for angioplasty, or to mount a stent over it if the catheter is intended to be used in a stent delivery 164 as shown in FIG.-5.

FIG.-6 schematically illustrates a rapid exchange mode of triple-profile balloon catheter 10. The proximal hub 42 has a proximal end 12 with proximal guidewire opening 22 and a strain relief sleeve 30. The guidewire 34 enters into the rapid exchange guidewire opening 18 which not at the proximal end 12, 42 but in a closer distance from the balloon 100 location on the wall of catheter shaft 26. This figure also shows a stent 164 crimp-mounted on the folded balloon skin 142.

FIG.-6 schematically illustrates an over-the-wire exchange mode of triple-profile balloon catheter 10. The proximal end of the catheter has a Y-connector 16 with a guidewire lumen opening 18 and an inflation/deflation opening 22. There no guidewire lumen opening on a wall of the catheter shaft 26. This figure also shows a stent 164 crimp-mounted on the folded balloon skin 142.

In one embodiment, the first average diameter 110 can be a substantially constant first diameter, and the second average diameter 112 can be a substantially constant second diameter, at least a portion of the intermediate segment 122 has a variable diameter.

In various embodiments of the present invention, (i) the first segment 110 of the balloon 100 has a parallel silhouette in its longitudinal cross-section, (ii) the first segment 110 of the balloon 100 has a non-parallel silhouette when it is inflated in its longitudinal cross-section, (iii) the second segment 112 of the balloon 100 has a parallel silhouette when it is inflated in its longitudinal cross-section, (iv ) the second segment 112 of the balloon 100 has a non-parallel silhouette in its longitudinal cross-section, (v) at least a portion of the intermediate segment 114 of the balloon 100 has a parallel silhouette in its longitudinal cross-section, (vi) at least a portion of the intermediate segment 114 of the balloon 100 has a non-parallel silhouette in its longitudinal cross-section when inflated, (vii) the intermediate segment 114 of the balloon 100 has a smooth profile, (viii) the intermediate segment 114 of the balloon 100 has a non-smooth profile, and the like.

The catheter shaft 26 has the distal end 28 with a guidewire 30 extending from the tip 14. A guidewire lumen 32 ends at the distal end of the catheter shaft 28 where the guidewire 34 is in place. In various embodiments, the inflation/deflation lumen 36 in the catheter shaft 26 includes an inflation-deflation aperture 24 positioned, inside the first segment 110 of the balloon chamber 40, inside the second segment 112 of the balloon chamber 40, inside the intermediate segment 114 of the balloon chamber 40, inside the balloon chamber 40 between the first segment 110 and the intermediate segment 114, inside the balloon chamber 40 between the second segment 112 and the intermediate segment 114. Both ends 102, 104 of the triple profile balloon 100 are bonded with bonding joints 138,140 on the catheter shaft 28, 28.

Significant elements of the triple-profile balloon 100 are the shapes of three segments 110, 112 and 114, with the three different profiles. The triple-profile balloon 100 has the single chamber 40 and has at least one inflation and deflation aperture 24 or opening connected to the inflation/deflation lumen 36. As illustrated in FIG. 1, the inflation-deflation lumen aperture 24 can be located in the second segment. 112. One reason for locating the inflation-deflation lumen aperture 24 in the second segment 112 is to first inflate the distal portion of the triple profile balloon 100 before other portions of the triple profile balloon 100, especially when the triple profile balloon 100 is used for stent 164 delivery and deployment. This distal location of the inflation-deflation aperture 24 may minimize or prevent sliding of the triple profile balloon 100 inside a vessel lumen during inflation of the balloon 100. However, the inflation-deflation aperture 24 can be located in any of the three segments 110, 112 or 114.

The proximal portion of the inflated triple-profile balloon 100 has the largest average diameter which can be of parallel profiles, and the distal portion of the inflated triple-profile balloon 100 has the smallest diameter that can have parallel profile. The intermediate segment 114 can have a progressively scaled non-parallel profile from the largest diameter at the first junction 116(b) between the proximal parallel zone 128 and the intermediate segment 132 to the smallest diameter at the second junction 118(c) between the distal parallel zone 130 and the intermediate segment 132 as shown in FIG. 2. The first radiopaque marker 120 can be on the catheter shaft 38 to coincide with a first balloon profile junction 116(b) between the first segment 110 and the intermediate segment 114 in order for the first radiopaque maker 120 to provide an indicate of the location of the first balloon profile junction 116(b) under fluoroscopy during angioplasty procedure. The second radiopaque marker 122 can be on the catheter shaft 38 to coincide with a second balloon profile junction 118(c) between the second segment 112 and the intermediate segment 114 so that the second radiopaque maker 122 indicates the location of the second balloon profile junction 118(c) under fluoroscopy during angioplasty procedure.

In addition to the first and second radiopaque markers 120 and 122, third and fourth radiopaque markers 124 and 126 can be included. The third radiopaque marker 124 is located in the proximal end of the triple profile balloon 100 to indicate its beginning under fluoroscopy during an angioplasty procedure. The fourth radiopaque marker 126 is located at the distal end of the triple profile balloon 100 to indicate the ending of the triple-profile balloon 100 under fluoroscopy during an angioplasty procedure. There can be four radiopaque markers 120, 122, 124 and 126 in the triple profile balloon 100 embodiment of FIG.-1. The first and second radiopaque markers 120 and 122, when placed between the proximal 102 and distal 104 ends of the triple profile balloon 100, have more critical functions and purposes than the third 124 and fourth 126 radiopaque markers. The position of the first and second radiopaque markers 120 and 122, as described above, in the middle portion of the triple profile balloon 100, follows the location of the first and second profile junctions 116 and 118 as the proportional lengths of the three different diameter portions of the triple profile balloon 100 are changed.

In one embodiment, the triple-profile balloon 100 provides a very fine tuned angioplasty balloon dilatation or PTCA in a more complex vessel anatomy, especially in a coronary artery anatomy. In certain segments of a coronary artery, the proximal portion has a larger diameter and the distal portion has a smaller diameter, whereas the middle portion may have a transitional diameter between the two dissimilar diameter portions. This type of vessel anatomy, typically, occurs in a vessel segment with a side-branch take-off, with a bifurcation or when the vessel segment is relatively long in length. A customary, conventional single diameter balloon has a mismatched balloon contour for this type of vessel anatomy. If a conventional balloon that matches the proximal large diameter portion of the vessel is selected, the proximal vessel portion may have an optimal dilatation, but the distal small diameter portion may be over-dilated causing a vessel injury or a dissection. If a conventional balloon is chosen to match the distal small diameter portion of the vessel is selected, the distal vessel portion may have an optimal dilatation, but the proximal large diameter segment may be under-dilated causing an incomplete result or cause a new problem. In a stent deployment scenario in this type vessel anatomy, if a balloon that matches the distal small vessel size, the proximal large diameter vessel segment would be under-dilated causing a mal-apposition and poor contact of the stent with to vessel wall. A mal-apposition of a drug-eluting stent could cause late post-stent complications.

Using the triple-profile balloon 116 with the first segment 110, second segment 112 and intermediate segment 114 that have different diameters, this type of coronary anatomy could optimally matched with the triple-profile balloon 100. Utilization of the triple-profile balloon 100 would result in anatomically optimal and safe effects in these vessel anatomies, with markedly reduced complications after coronary interventions. The profile of the expanded triple-profile balloon 100 is better matched to the natural vessel anatomy in these complex portions of a coronary artery segments or other vascular anatomies than the customary simple balloon with single diameter profile. Use of the triple-profile balloon 100 prevents vessel dissections or stent mal-appositions and reduces the acute and long term co-morbidity of coronary intervention, whether used for a balloon angioplasty, stent delivery, pre-dilatation or post-dilation. If the triple-profile balloon 100 is used for stent delivery and deployment in these vessel anatomies, the complication and restenosis rate may also be reduced.

In one embodiment, the first segment 110 could have a length of about one third of an effective total length of the balloon 100. In various embodiments, (i) the first segment 100 has a length greater than one third of the effective total length of the balloon 100, (ii) the first segment 110 has a length less than one third of the effective total length of the balloon 100, (iii) the second segment 112 has a length about one third of an effective total length of the balloon 100, (iv) the second segment 112 has a length greater than one third of the effective total length of the balloon 100, (v) the second segment 112 has a length less than one third of the effective total length of the balloon 100, (vi) the intermediate segment 114 has a length about one third of an effective total length of the balloon 100, (vii) the intermediate segment 114 has a length greater than one third of the effective total length of the balloon 100, (viii) the intermediate segment 114 has a length less than one third of the effective total length of the balloon 100.

In various embodiments, the first segment 110 has a length larger than a length of the second segment 114, the second segment 114 has a length larger than a length of the first segment 110, the lengths of the first and the second segments 110 and 112 are about the same, the intermediate segment 114 has a length larger than a length of the first or second segments 110 and 112, the intermediate segment 114 has a length smaller than a length of the first or second segments 110 and 112, the intermediate segment 114 has a length about the same as a length of the first or second segments 110 and 112.

In FIG. 2, the vertical dotted lines (a), (b), (c) and (d) are drawn as demarcations between the first, second and intermediate segments 110, 112 and 114. The first two demarcation lines (a) and (b) define the beginning and ending of the first segment 110 of the inflated triple-profile balloon 100. The last two demarcation lines (c) and (d) define the beginning and ending of the second segment 112. The middle two demarcation lines (b) and (c) define the intermediate segment 114. The second demarcation line (b) coincides with the first transitional junction 116 between the first segment 110 and the intermediate segment 114. The second demarcation line (b) can also coincide with the location of first radiopaque marker 120. Therefore, the second demarcation line (b) can coincide with both the location of first radiopaque marker 120 and the first transitional junction 116 between the first segment 110 and the intermediate segment 114. The third demarcation line (c) coincides with the location of the second transitional junction 118 between the second segment 112 and the intermediate segment 114. The third demarcation line (c) can also coincide with the location of second radiopaque marker 122. Therefore, the third demarcation line (c) can coincide with both the location of second radiopaque marker 122 and the second transitional junction 118 between the second segment 112 and the intermediate segment 114. Similarly, the first demarcation line (a) defines the beginning of the effective length of the balloon 100 and the first segment 110 and coincides with the location of the third radiopaque marker 124. The fourth demarcation line (d) defines the ending of an effective length of the balloon 100 and coincides with the location of the fourth radiopaque marker 126 and the distal end of the second segment 112. In FIG. 2, the distal aperture 24 of the inflation/deflation lumen 32 is shown as being located between the third (c) and fourth (d) demarcation lines of the triple profile balloon 100.

FIG. 3A is a longitudinal cross-section of the triple-profile balloon 100 of FIG.-2. A stent 150 is shown as expanded by the inflated triple-profile balloon 100. Because the stent 150 is passively expanded by the triple-profile balloon 100, after being crimped on the folded triple-profile balloon for delivery, the expanded stent 150 has the identical longitudinal profile as the inflated triple-profile balloon 100 underneath. Although FIG. 3A illustrates how the triple-profile balloon profile shapes the profile of the expanded stent 150 when the triple-profile triple profile balloon 100 is used as a stent delivery vehicle, the profile of the triple-profile balloon 100 also shapes a vessel wall the same way when the triple-profile balloon 100 is used in a vessel lumen for balloon dilatation.

The catheter shaft 26 ends at the distal end 14 on the right. The longitudinal cross-section of the triple profile-balloon catheter 100 can contain the inflation/deflation and guidewire lumens 36 and 34, as described above. Although the guidewire lumen 32 traverses through the end of the catheter shaft 26, the inflation/deflation lumen 36 ends at the distal aperture 24 or opening that connects into the closed chamber 40 of the triple-profile balloon 100, which in this illustrated embodiment, is in the second segment 112. As shown in the FIG. 3A embodiment, there are the dotted demarcation lines (a), (b), (c) and (d) are shown as corresponding with radiopaque markers 124, 120, 122 and 126, respectively, that divide triple profile balloon 100 into three balloon profile zones; a proximal parallel zone 152 at the first segment 110, a distal parallel zone 154 at the second segment 112 and an intermediate transitional zone 156 at the intermediate segment 114. These radiopaque markers 124, 120, 122 and 126 will become critical reference point under fluoroscopy to indicate where the three profile zones of the triple-profile balloon 100 during a balloon angioplasty procedure. Likewise, the same radiopaque markers 124, 120, 122 and 126 play the critical role of indicating where the stent 100 should be deployed into the expanded shape in the coronary vessel anatomy during delivery phase of a stenting procedure. When the stent 150 is delivered by the triple-profile balloon 100 in the right location and deployed with pressure inflation of the balloon 100, the stent 150 is expanded and molded into place by the shape and profile of the inflated triple-profile balloon 100. The three profile zones of the expanded delivery triple-profile balloon 100 correlate with the three profile zones, respectively, of the expanded stent 150 that is delivered and deployed by the triple-profile balloon 100. One cannot tell under fluoroscopy, during a stent or angioplasty procedure, where are the demarcation of three profile zones of the triple-profile balloon 100 or the stent crimp-mounted on the triple-profile balloon 100, without the radiopaque makers 124, 120, 122 and 126. If there are no first and second radiopaque marker 120 and 122 as references, one cannot place the stent 100 at the right anatomic location where a stent 150 should be deployed.

In FIG. 3B, a longitudinal cross-sectional view of the expanded stent 150, isolated from FIG. 3A is provided. As indicated above, the expanded stent 150 is passively shaped by the shape of the pressure inflated triple-profile balloon configuration 150 of FIG. 3A, which is used as a stent delivery balloon. The shape of the expanded stent 150 profile in FIG. 3A is one of the two critical purposes of the shape of the triple-profile balloon 100. Another other purpose is to dilate and shape a diseased vessel that has dissimilar diameter profile with a larger proximal diameter and a smaller distal diameter. The passively expanded stent 150, with three profile zones as shown in FIG.-3B, take after the shape of three diameter profile zones of the inflated triple-profile balloon 150 in FIG. 3A. The expanded stent 150, passively shaped by the triple-profile balloon 100, has a proximal end 158, a distal end 160 and an open lumen 162 that is shaped in place by the triple-profile balloon 100. The expanded stent 150 has a proximal parallel zone 152, a distal parallel zone 154 and an intermediate transitional zone 156, which are shaped by the three profile zones 110, 112 and 114, respectively, of the triple-profile balloon 100. Both the shape and internal diameter of the expanded stent 150 is formed by the shape and diameter of the inflated triple-profile balloon 100, in FIG. 3A.

FIG. 4 is a schematic external view of the deflated and folded triple-profile balloon 100 similar to that shown in FIGS. 1, 2 and 3A. With the skin of the triple-profile balloon 100 folded into a low profile form, this triple-profile balloon 100 is ready to be used as a simple angioplasty dilatation balloon or as a triple-profile balloon 100 to deliver the stent 150. Although the triple-profile balloon 100 has its unique profile and shape when inflated, the triple-profile balloon 100 may have little or no resemblance of the triple-profile shape 100 when it is deflated and folded, depending on what kind of material is used for fabrication of the triple-profile balloon 100.

As used for angioplasty, the triple-profile balloon 100 can be applied to a simple balloon dilatation, pre-dilatation, post-dilatation, as well as stent delivery. When used for stent delivery, a stent 150 is crimp-mounted over the folded skin 108 of the triple-profile balloon 100 for delivery and deployment of the stent 150. In this external view of the triple-profile balloon 100, with the folded skin 108, the four radiopaque markers 120,1222, 124 and 126 of the triple-profile balloon 100 are not shown. In FIG. 4, the proximal 102 and distal 104 ends of the triple-profile balloon 100 show the triple-profile balloon attachment bonding joints 138 and 140. A guidewire 34 is in place and extends from the distal end 14 of the catheter shaft 28.

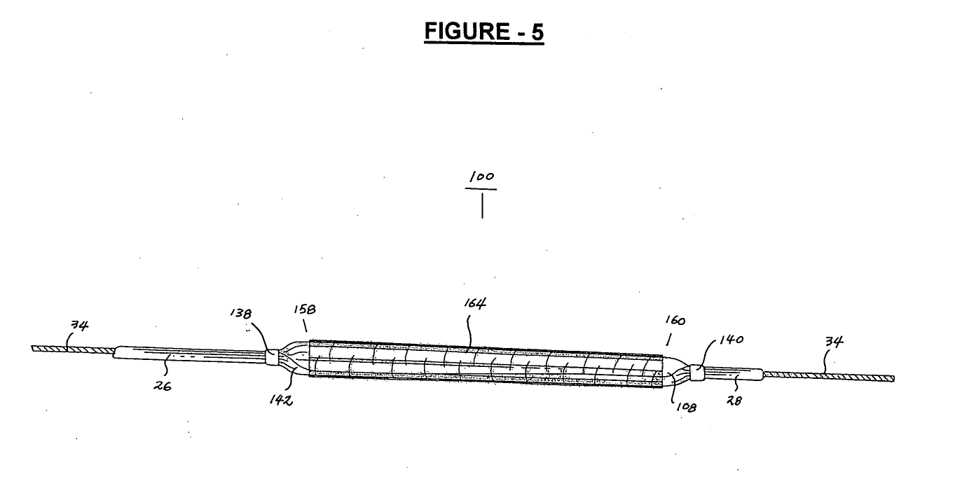

FIG. 5 is a schematic drawing to illustrate how a stent 164 is crimp-mounted on a deflated and folded 142 tripe-profile balloon 100 for stent delivery. The stent 164 is smoothly and evenly crimped tightly for delivery on a deflated and folded skin 142 of the triple-profile balloon 100 of FIGS. 1, 2 and 3A. The folded triple-profile balloon 142 underneath the crimp-mounted stent 164 is similar to the one illustrates in FIG. 4. The proximal 158 and distal 160 ends of the stent 164 mounted on the triple-profile balloon 100 are inside of the third 124 and fourth 126 radiopaque markers, respectively, which are underneath the folded skin 142 of the triple-profile balloon 100 and are not shown in FIG. 5. This crimp-mounted stent 164 on the triple-profile balloon 100 is ready to be introduced into a vessel for stent delivery and deployment. Typically, a stent 164 crimp-mounted tightly on a folded triple-profile balloon 100 is inserted into target artery and advanced to the lesion site, where the stent 164 is needed. With the stent 164 is expanded and deployed by inflating the triple-profile balloon 100 at the lesion site, the balloon 100 is withdrawn from the vessel site, leaving only the expanded stent 150 in place. Once firmly deployed in a vessel site, the stent 150 cannot be retrieved by a percutaneous method.

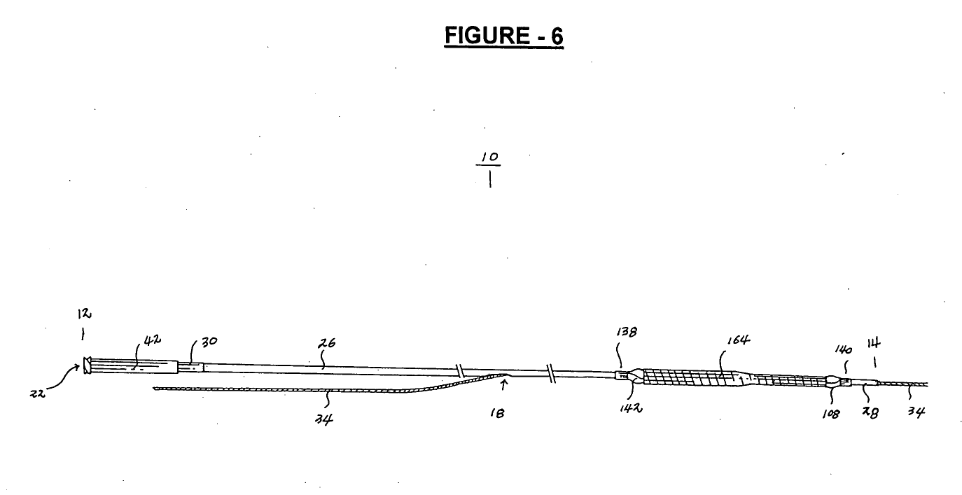

FIG. 6 is a schematic illustration of how the triple profile-balloon catheter 100 can be adapted to a rapid-exchange catheter system. This drawing also illustrates how the same triple-profile balloon catheter 100 can be used as a stent delivery system. A proximal end 12 of the triple-profile balloon catheter 10 has an opening for inflation-deflation lumen 22, and a connector for an inflation-deflation adopter 42. A distal end 14 of the catheter 10 is shown. The distal end of the inflation/deflation lumen 24 ends inside the balloon lumen 40 for inflating and deflating of the triple-profile balloon 100. The distal opening of the inflation/deflation lumen 24 is not shown in this drawing because the aperture 24 is buried inside the folded skin of the triple-profile balloon 100 and crimp-mounted stent 164 over the triple-profile balloon 100. The catheter shaft 26 starts from the distal end of the proximal connector 42, where a strain-relief sleeve 30 is located, and ends at the tip 14 of the triple profile-balloon catheter 10. A proximal guidewire opening 18 is between the proximal end 12 of the triple-profile balloon catheter 10 and the balloon segment 100. The guidewire lumen 32 runs through the catheter shaft 26, ending at the distal end 14 of the balloon catheter 10, where a guidewire 34 is protruding through the distal guidewire opening 22. Although FIG. 6 has a stent 164 crimped in a stepped-down profile of the folded triple profile balloon 142, a stent 164 crimp-mounted on the triple-profile balloon 100 may have a smooth outer contour without a stepped shape, depending on what kind of material is used for the fabrication of the triple-profile balloon 100.

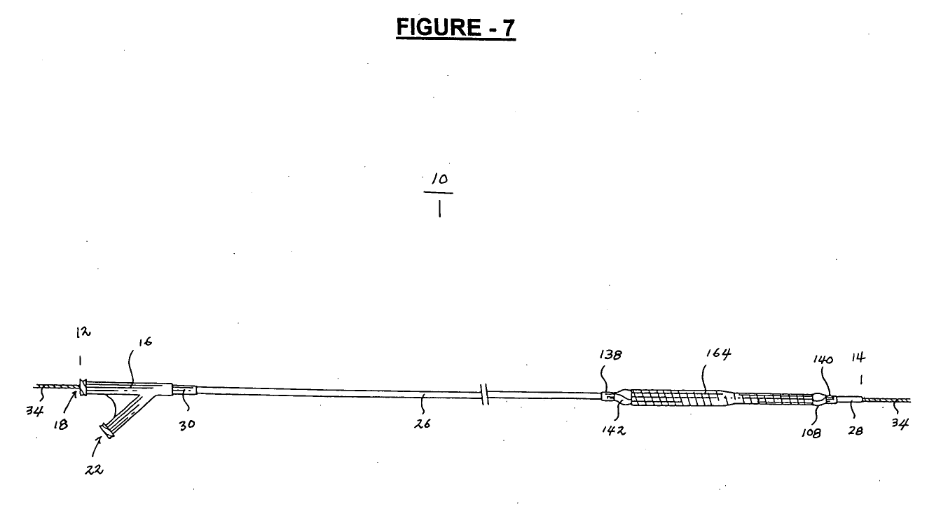

FIG. 7 is a schematic illustration of how the triple-profile balloon catheter 10 can be adapted to an over-the-wire catheter system. FIG. 7 also illustrates how the same triple profile balloon catheter 10 can be used as a stent delivery system. The proximal end 12 of the triple-profile balloon catheter 10 has a Y-connector 16 for openings of an inflation/deflation lumen 18 and the guidewire lumen 22. Lumens 32 and 36 run through most of the entire length of the catheter shaft 26. The guidewire lumen 32 runs through the entire length of the catheter shaft 26, from tip 12 to tip 14, with a guidewire 34 protruding from both ends 22 and 20 of the triple-profile balloon catheter 10. The proximal end of the inflation/deflation lumen 36 starts from the side arm of the Y-connector 16 and ends inside the balloon chamber 40. The distal opening of the inflation/deflation lumen 36 is not shown in FIG. 7 because the aperture 24 is buried inside the folded skin 142 of the triple-profile balloon 100 and the crimp-mounted stent 164. The catheter shaft 26 starts from the distal end of the proximal Y-connector 16, where there is a short strain relief sleeve 30, and ends at the tip 12 of the triple-profile balloon catheter 10. Although FIG. 7 shows a stent 164 crimped in a stepped-down profile on the folded triple-profile balloon 100, a stent 164 crimp-mounted on the triple-profile balloon 100 may have a smooth outer contour without a stepped shape, depending on what kind of material is used for fabrication of the triple-profile balloon 100.

The triple-profile balloon catheter 100 can be used for angioplasty, used in the vascular system in the body including the coronary artery and vein, carotid and cerebral artery and vein, renal artery and vein, peripheral vascular artery and vein, aorta, or superior and inferior vena cava, and the like. The triple-profile balloon catheter 100 can be used in other tubular anatomic body organ anatomy other than a vascular system.

In one embodiment of the present invention, the triple profile balloon catheter 100 of present invention is a specially designed balloon for use in specific anatomic characteristics of bifurcation or side-branch origin lesions of vessels. A bifurcation in coronary anatomy is created when a main branch gives rise to a side branch. A side-branching of coronary anatomy creates a hub that is connected to three separate segments of the vessel: a main branch proximal to the branching point, a new side branch distal to the branching point and an extension of the main branch distal to the branching point. The branching point becomes a bifurcation. Alternately, a main branch may bifurcate into two similar sized branches that are smaller in caliber than the main branch. At the hub of bifurcation, the two bifurcated branches create a saddle. In other words, a bifurcation is formed when an artery divides into two distal branches. Therefore, in a conceptual sense, a side-branch origin is a bifurcation, and a bifurcation is associated with a side-branch origin. This concept is repeated in various forms through out the disclosure of the present invention. For this reason, side-branch origin and bifurcation are used to describe the same or similar anatomic characteristics herein.

The anatomy of a bifurcation may have as many as three different vessel diameters associated with a bifurcation point. When an atherosclerotic lesion develops at a bifurcation or saddle, one, two or all three branches could be involved with atherosclerotic plaques. These three branches may have three different vessel diameters. Furthermore, an angle at which a side branch takes off from the main branch also has a wide range of variations.

The most critical basic element for designing a stenting system for the bifurcation or side-branch origin lesions is a specially designed angioplasty balloon. Without a well-suited angioplasty balloon design, a specially designed bifurcation stent cannot be properly or successfully implanted in a bifurcation or side-branching origin lesions. A stent is a passive device delivered, expanded and molded inside a vessel lumen, by an angioplasty balloon. Because coronary bifurcations have variable sets of complex anatomic characteristics as described in the preceding descriptions, a simple single diameter balloon would not be adequate for a stent delivery system for lesions in a bifurcation or side-branch origin, because such lesion straddles proximal large vessel and distal small vessels of two different sizes. An angioplasty or stent delivery balloon is an elongated and enclosed tubular structure. A stent is delivered, expanded and molded into an elongated tubular structure inside a lesion site by the external shape of a stent delivery balloon underneath, when the balloon is inflated with high-pressurized saline. A typical stent is expanded and deployed with a nominal inflating balloon pressure of 8-10 ATM (atmospheric pressure), but some balloons can tolerate a pressure as much as 20 ATM or more.

To adapt to the especially complex set of anatomic characteristics of the bifurcation and side-branch origin of coronary artery, the triple-profile balloon tube 100 has a triple-profile inflated balloon contour. The radiopaque markers 120 and 122 can be located at or near the transition junction 116 the first segment 110 and the intermediate segment 114, and at or near the transition junction 118 between the second segment 112 and the intermediate segment 114, respectively.

The triple-profile balloon 100 can be used for a variety of different applications, including but not limited to balloon angioplasty and stent delivery in vascular and tubular anatomy. In a stent implant procedure, particularly in complex anatomic environment like in bifurcation lesions, a pre-stent and post-stent balloon dilatation of the stenotic lesion is often a pre-requisite. The triple-profile balloon 100 is designed for balloon angioplasty alone.

If all three of proximal main branch and two distal side branches at or near a bifurcation are affected by an atherosclerotic lesion, then all three vessel segments at the bifurcation need angioplasty and stenting. In this scenario, all three vessels may have three different vessel diameters. The triple-profile balloon 100 can be utilized in this situation. Two separate triple-profile balloons 100, with proper diameter combination of the first segment 110 and the second segment 112 could conceivably produce the desired outcome at such bifurcation lesions. A properly fitting first triple-profile balloon 100 delivers and deploys a triple profile stent into the first side branch. The proximal large portion of the stent is matched with the proximal large main branch, and the distal smaller diameter portion of the stent is matched with the smaller side-branch for stent deployment. When a stent is deployed in a side-branch at a bifurcation, the strut network of the expanded stent would blocks the lumen of the other un-stented branch by the stent struts of an intermediate portion of the stent 100.

In this inevitable aftermath of a bifurcation stenting, a jailbreak is performed to open up a circular hole in a cell of the intermediate portion of the stent that blocks the vessel lumen. A jailbreak is performed by inserting a deflated balloon 100 over a guidewire passed through the jail-blocking stent struts and inflating the balloon 100 inside a stent cell to make the cell a round hole to match the lumen of the blocked artery. If only the first side-branch needs for stenting, the angioplasty procedure ends with one stent implant and one jail-breaking here.

When stenting of the second branch of the bifurcation is needed, a second triple-profile stent that matches the vessel lumen is deployed in the second side branch, repeats the similar procedural steps, as the first side-branch stenting. At this point, the struts of the intermediate portion of the second stent now block the orifice of the first side-branch which was already stented. This requires another, second, balloon jailbreak of the stent cell of the transitional portion of the second stent, to open up the blocked orifice of the first side-branch that received the first stent. When the second triple profile stent is deployed, the main branch proximal to the bifurcation has two over-lapping stent segments.

As described in these angioplasty and stenting scenarios, it will be appreciated how the triple-profile balloon 100 could play a critical role in a successful angioplasty and stenting in the bifurcation anatomy. Any specially designed bifurcation stent cannot work well, unless a specially designed and effective stent delivery balloon, such as the triple-profile balloon 100 is used to support it. This is because a stent is a passive device that depends on a balloon based angioplasty balloon catheter system.

The foregoing description of a preferred embodiment of the invention has been presented for purposes of illustration and description. It is not intended to be exhaustive or to limit the invention to the precise forms disclosed. Obviously, many modifications and variations will be apparent to practitioners skilled in this art. It is intended that the scope of the invention be defined by the following claims and their equivalents.

1. An angioplasty balloon catheter system, comprising:
a catheter shaft with an inflation/deflation lumen; and
a balloon located at a distal section of the catheter shaft, the balloon having a first segment in a proximal portion of the balloon, and a second segment in a distal portion of the balloon, the first and second segments being coupled by an intermediate segment with a length greater than 3 mm, the first segment having a first average diameter, the second segment having a second average diameter that is less than the first average diameter, the balloon having a single chamber coupled to the inflation/deflation lumen.

2. The catheter of claim 1, wherein the first average diameter is a substantially constant first diameter, and the second average diameter is a substantially constant second diameter.

3. The catheter of claim 1, wherein the first average diameter is a substantially constant first diameter.

4. The catheter of claim 1, wherein the second average diameter is a substantially constant second diameter.

5. The catheter of claim 1, wherein at least a portion of the intermediate segment has a variable diameter.

6. The catheter of claim 1, wherein a radiopaque marker is located at or near the transition junction between the first segment and the intermediate segment.

7. The catheter of claim 6, wherein the radiopaque marker is located inside the balloon chamber.

8. The catheter of claim 1, wherein a radiopaque marker is located at or near the transition junction between the second segment and the intermediate segment.

9. The catheter of claim 8, wherein the radiopaque marker is located inside the balloon chamber.

10. The catheter of claim 1, wherein a first radiopaque marker is located at or near the transition junction between the first segment and the intermediate segment, and a second radiopaque marker is located at or near the transition junction between the second segment and the intermediate segment.

11. The catheter of claim 10, wherein the first and second radiopaque markers are located inside the balloon chamber.

12. The catheter of claim 1, wherein the first segment has a length of about one third of an effective total length of the balloon.

13. The catheter of claim 12, wherein the first segment has a length greater than one third of the effective total length of the balloon.

14. The catheter of claim 12, wherein the first segment has a length less than one third of the effective total length of the balloon.

15. The catheter of claim 1, wherein the second segment has a length about one third of an effective total length of the balloon.

16. The catheter of claim 15, wherein the second segment has a length greater than one third of the effective total length of the balloon.

17. The catheter of claim 15, wherein the second segment has a length less than one third of the effective total length of the balloon.

18. The catheter of claim 1, wherein the intermediate segment has a length about one third of an effective total length of the balloon.

19. The catheter of claim 18, wherein the intermediate segment has a length greater than one third of the effective total length of the balloon.

20. The catheter of claim 18, wherein the intermediate segment has a length less than one third of the effective total length of the balloon.

21. The catheter of claim 1, wherein the first segment of the balloon has a parallel silhouette in its longitudinal cross-section.

22. The catheter of claim 21, wherein the first segment of the balloon has a non-parallel silhouette in its longitudinal cross-section.

23. The catheter of claim 1, wherein the second segment of the balloon has a parallel silhouette in its longitudinal cross-section.

24. The catheter of claim 23, wherein the second segment of the balloon has a non-parallel silhouette in its longitudinal cross-section.

25. The catheter of claim 1, wherein at least a portion of the intermediate segment of the balloon has a parallel silhouette in its longitudinal cross-section.

26. The catheter of claim 25, wherein at least a portion of the intermediate segment of the balloon has a non-parallel silhouette in its longitudinal cross-section.

27. The catheter of claim 1, wherein the intermediate segment of the balloon has a smooth profile.

28. The catheter of claim 27, wherein the intermediate segment of the balloon has a non-smooth profile.

29. The catheter of claim 1, wherein the balloon has a single internal lumen.

30. The catheter of claim 1, wherein the balloon has a single internal chamber.

31. The catheter of claim 1, wherein the balloon is made of a material selected from a polymer, non-polymer or composite material.

32. The catheter of claim 1, wherein the inflation/deflation lumen in the catheter shaft includes an inflation-deflation aperture positioned inside the first segment of the balloon chamber.

33. The catheter of claim 1, wherein the inflation/deflation lumen in the catheter shaft includes an inflation-deflation aperture positioned inside the second segment of the balloon chamber.

34. The catheter of claim 1, wherein the inflation/deflation lumen in the catheter shaft includes an inflation-deflation aperture positioned inside the intermediate segment of the balloon chamber.

35. The catheter of claim 1, wherein the inflation/deflation lumen in the catheter shaft includes an inflation-deflation aperture positioned inside the balloon chamber between the first segment and the intermediate segment of the balloon.

36. The catheter of claim 1, wherein the inflation/deflation lumen in the catheter shaft includes an inflation-deflation aperture positioned inside the balloon chamber between the second segment and the intermediate segment of the balloon.

37. The catheter of claim 1, wherein a radiopaque marker is positioned at a proximal end of the balloon.

38. The catheter of claim 1, wherein a radiopaque marker is positioned at a distal end of the balloon.

39. The catheter of claim 1, wherein the balloon catheter is configured to be included in an over-the-wire catheter system.

40. The catheter of claim 1, wherein the balloon catheter is configured to be included in a rapid-exchange catheter system.

41. The catheter of claim 1, wherein the balloon catheter is configured for angioplasty.

42. The catheter of claim 1, wherein the first segment has a length larger than a length of the second segment.

43. The catheter of claim 1, wherein the second segment has a length larger than a length of the first segment.

44. The catheter of claim 1, wherein the lengths of the first and the second segments are about the same.

45. The catheter of claim 1, wherein the intermediate segment has a length larger than a length of the first or second segment.

46. The catheter of claim 1, wherein the intermediate segment has a length smaller than a length of the first or second segment.

47. The catheter of claim 1, wherein the intermediate segment has a length about the same as a length of the first or second segment.

48. The catheter of claim 1, wherein the angioplasty balloon catheter system is configured for use in the vascular system in the body including the coronary artery and vein, carotid and cerebral artery and vein, renal artery and vein, peripheral vascular artery and vein, aorta, or superior and inferior vena cava.

49. The catheter of claim 1, wherein the angioplasty balloon catheter system is configured for use in a tubular anatomic body organ other than a vascular system.

50. The catheter of claim 1, wherein the balloon has a smooth transition junction between the first segment and the intermediate segment.

51. The catheter of claim 1, wherein the balloon has a smooth transition junction between the intermediate segment and the second segment.

52. The catheter of claim 1, wherein the intermediate segment has a smooth transition silhouette between the first and second diameters.

53. The catheter of claim 1, wherein the intermediate segment has a straight transition silhouette between the first and second diameter when the balloon is nominally inflated.

54. The catheter of claim 1, wherein the intermediate segment has a non-parallel transition silhouette between the first and second diameter when the balloon is nominally inflated.

55. A triple profile balloon catheter, comprising:
a catheter shaft with an inflation/deflation lumen and a guidewire lumen;
a balloon located at a distal section of the catheter shaft, the balloon having a first segment in a proximal portion of the balloon, and a second segment in a distal portion of the balloon, the first and second segments being coupled by an intermediate segment with a length greater than 3 mm, the first segment having a first average diameter, the second segment having a second average diameter that is less than the first average diameter; and
a first radiopaque marker positioned at a transition junction between the first segment and the intermediate segment of the balloon.

56. The catheter of claim 55, wherein a second radiopaque marker is positioned at a transition junction between the second segment and the intermediate segment of the balloon.

57. The catheter of claim 55, wherein the first radiopaque marker is configured for use as a reference marker of the transition junction between the first segment and the intermediate segment of the balloon.

58. The catheter of claim 56, wherein the second radiopaque marker is configured for use as a reference marker of the transition junction between the second segment and the intermediate segment of the balloon.

59. The catheter of claim 55, wherein the first radiopaque marker is configured for use as a reference marker for a vascular anatomy under fluoroscopy.

60. The catheter of claim 55, further comprising:
a second radiopaque marker positioned at a transition junction between the second segment and the intermediate segment of the balloon.

61. The catheter of claim 60, wherein the second radiopaque marker in the balloon is configured for use as a reference marker of the transition junction between the second segment and the intermediate segment.

62. The catheter of claim 60, wherein the second radiopaque marker is configured for use as a reference marker for a vascular anatomy under fluoroscopy.

63. A triple profile balloon catheter, comprising:
a catheter shaft with an inflation/deflation lumen; and
a balloon located at a distal section of the catheter shaft, the balloon having a first segment in a proximal portion of the balloon, and a second segment in a distal portion of the balloon, the first and second segments being coupled by an intermediate segment with a length greater than 3 mm, the first segment having a first average diameter, the second segment having a second average diameter that is less than the first average diameter; and
a first radiopaque marker positioned at a transition junction between the second segment and the intermediate segment of the balloon.

64. The catheter of claim 63, wherein the first radiopaque marker in the balloon is configured for use as a reference marker of the transition junction between the second segment and the intermediate segment.

65. The catheter of claim 63, wherein the first radiopaque marker is configured for use as a reference marker for a vascular anatomy under fluoroscopy.

66. The catheter of claim 63, further comprising:
a second radiopaque marker positioned at a transition junction between the first segment and the intermediate segment.

67. The catheter of claim 66, wherein the second radiopaque marker in the balloon is configured for use as a reference marker of the transition junction between the first segment and the intermediate segment.

68. The catheter of claim 66, wherein the second radiopaque marker is configured for use as a reference marker for a vascular anatomy under fluoroscopy.

69. A triple profile balloon catheter, comprising:
a catheter shaft with an inflation/deflation lumen; and
a balloon located at a distal section of the catheter shaft, the balloon having a first segment in a proximal portion of the balloon, and a second segment in a distal portion of the balloon, the first and second segments being coupled by an intermediate segment, the first segment having a first average diameter, the second segment having a second average diameter that is less than the first average diameter;
a first radiopaque marker positioned at or near a transition junction between the first segment and the intermediate segment; and
a second radiopaque marker positioned at or near a transition junction between the second segment and the intermediate segment of the balloon.

70. The catheter of claim 69, wherein the first radiopaque marker in the balloon is configured for use as a reference marker of the transition junction between the first segment and the intermediate segment.

71. The catheter of claim 69, wherein the first radiopaque marker is configured for use as a reference marker for a vascular anatomy under fluoroscopy.

72. The catheter of claim 69, wherein the second radiopaque marker in the balloon is configured for use as a reference marker of the transition junction between the second segment and the intermediate segment.

73. The catheter of claim 69, wherein the second radiopaque marker is configured for use as a reference marker for a vascular anatomy under fluoroscopy.

74. A triple profile balloon catheter, comprising:
a catheter shaft with an inflation/deflation lumen and a guidewire lumen;
a balloon located at a distal section of the catheter shaft, the balloon having a first segment in a proximal portion of the balloon, and a second segment in a distal portion of the balloon, the first and second segments being coupled by an intermediate segment, the first segment having a first average diameter, the second segment having a second average diameter that is less than the first average diameter, the balloon having a single chamber coupled to the inflation/deflation lumen; and
at least a first radiopaque marker positioned at or near a transition junction between the first segment and the intermediate segment or at or near a transition junction between the second segment and the intermediate segment of the balloon.

75. The catheter of claim 74, wherein the first radiopaque marker in the balloon is configured for use as a reference marker of the transition junction between the first segment and the intermediate segment or the transition junction between the second segment and the intermediate segment of the balloon.

76. The catheter of claim 74, wherein the first radiopaque marker is configured for use as a reference marker for a vascular anatomy under fluoroscopy.

77. The catheter of claim 74, further comprising:
a second radiopaque marker located at or near a transition junction between the first segment and the intermediate segment or at or near a transition junction between the second segment and the intermediate segment of the balloon.

78. The catheter of claim 77, wherein the second radiopaque marker is configured for use as a reference marker of the transition junction between the first segment and the intermediate segment or the transition junction between the second segment and the intermediate segment of the balloon.

79. The catheter of claim 74, wherein the first radiopaque marker is configured for use as a reference marker for a vascular anatomy under fluoroscopy.

80. The catheter of claim 77, wherein the second radiopaque marker is configured for use as a reference marker for a vascular anatomy under fluoroscopy.

81. A triple profile balloon catheter, comprising:
a catheter shaft with an inflation/deflation lumen and a guidewire lumen; and
a balloon located at a distal section of the catheter shaft, the balloon having a first segment in a proximal portion of the balloon, and a second segment in a distal portion of the balloon, the first and second segments being coupled by an intermediate segment, the first, second and intermediate segments each having a different profile, the balloon having a single chamber coupled to the inflation/deflation lumen.

82. The catheter of claim 81, further comprising:
a first radiopaque marker positioned at a transition junction between the first segment and the intermediate segment.

83. The catheter of claim 82, wherein the first radiopaque marker in the balloon is configured for use as a reference marker of the transition junction between the first segment and the intermediate segment.

84. The catheter of claim 82, wherein the first radiopaque marker is configured for use as a reference marker for a vascular anatomy under fluoroscopy.

85. The catheter of claim 82, further comprising:
a second radiopaque marker positioned at a transition junction between the second segment and the intermediate segment.

86. The catheter of claim 85, wherein the second radiopaque marker in the balloon is configured for use as a reference marker of the transition junction between the second segment and the intermediate segment.

87. The catheter of claim 85, wherein the second radiopaque marker is configured for use as a reference marker for a vascular anatomy under fluoroscopy.

88. An angioplasty balloon catheter system, comprising:
a catheter shaft with an inflation/deflation lumen; and
a balloon located at a distal section of the catheter shaft, the balloon having a first segment in a distal portion of the balloon, and a second segment in a proximal portion of the balloon, the first and second segments being coupled by an intermediate segment with a length greater than 3 mm, the first segment having a first average diameter, the second segment having a second average diameter that is less than the first average diameter, the balloon having a single chamber coupled to the inflation/deflation lumen.

89. The catheter of claim 88, further comprising:
a first radiopaque marker positioned at a transition junction between the first segment and the intermediate segment.

90. The catheter of claim 89, wherein the first radiopaque marker in the balloon is configured for use as a reference marker of the transition junction between the first segment and the intermediate segment.

91. The catheter of claim 89, wherein the first radiopaque marker is configured for use as a reference marker for a vascular anatomy under fluoroscopy.

92. The catheter of claim 89, further comprising:
a second radiopaque marker positioned at a transition junction between the second segment and the intermediate segment.

93. The catheter of claim 92, wherein the second radiopaque marker in the balloon is configured for use as a reference marker of the transition junction between the second segment and the intermediate segment.

94. The catheter of claim 92, wherein the second radiopaque marker is configured for use as a reference marker for a vascular anatomy under fluoroscopy.

95. A triple profile balloon catheter, comprising:
a catheter shaft with an inflation/deflation lumen and a guidewire lumen; and
a balloon located at a distal section of the catheter shaft, the balloon having a first segment in a distal portion of the balloon, and a second segment in a proximal portion of the balloon, the first and second segments being coupled by an intermediate segment, the first, second and intermediate segments each having a different profile, the balloon having a single chamber coupled to the inflation/deflation lumen.

96. The catheter of claim 95, further comprising:
a first radiopaque marker positioned at a transition junction between the first segment and the intermediate segment.

97. The catheter of claim 96, wherein the first radiopaque marker in the balloon is configured for use as a reference marker of the transition junction between the first segment and the intermediate segment.

98. The catheter of claim 96, wherein the first radiopaque marker is configured for use as a reference marker for a vascular anatomy under fluoroscopy.

99. The catheter of claim 96, further comprising:
a second radiopaque marker positioned at a transition junction between the second segment and the intermediate segment.

100. The catheter of claim 99, wherein the second radiopaque marker in the balloon is configured for use as a reference marker of the transition junction between the second segment and the intermediate segment.

101. The catheter of claim 99, wherein the second radiopaque marker is configured for use as a reference marker for a vascular anatomy under fluoroscopy.
